(19) United States
(12) Patent Application Publication
(10) Pub. No.: US 2010/0076407 A1
(43) Pub. Date: Mar. 25, 2010

(54) CATHETERS FOR USE IN THE SUBARACHNOID SPACE AND THE LIKE

(76) Inventor: Lawrence Scott Ring, Valencia, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/234,396

(22) Filed: Sep. 19, 2008

(51) Int. Cl. A61M 25/098

(52) U.S. Cl. 604/529

(57) ABSTRACT

A catheter including an abutment located within the catheter body lumen.

### BACKGROUND

1. Field of Inventions

The present inventions relate generally to catheters that may be used to, for example, deliver medication to the subarachnoid space.

2. Description of the Related Art

Implantable infusion devices have been used to provide patients with a medication or other substance (collectively “infusible substance”) and frequently include an implantable pump and a catheter. A reservoir stores the infusible substance within the pump and, in some instances, implantable pumps are provided with a fill port that allows the reservoir to be transcutaneously filled (and/or re-filled) with a hypodermic needle. The reservoir is coupled to a fluid transfer device within the pump which is, in turn, connected to an outlet port. The catheter, which has at least one outlet, may be connected to the outlet port. As such, the infusible substance may be transferred from the reservoir to the target body region by way of the fluid transfer device and catheter.

The delivery of infusible substance into the subarachnoid space around the spinal cord or brain requires the use of catheters that are relatively long, thin and soft. While such mechanical properties are necessary and useful, they result in catheters that lack the structural rigidity required for the insertion process. As such, a stylet may be temporarily positioned within the catheter lumen and used to push the distal portion of the catheter to the target location. The distal portion of the catheter frequently includes the fluid outlet(s) and, accordingly, the distal portion also includes a visualization marker that may be observed through the use of suitable instrumentalities so that the fluid outlet(s) may be precisely positioned. The marker may be in the form of one or more thin marker rings that are carried on the distal portion of the catheter, or a marker tip that is mounted on the distal end of the catheter. Radiopaque markers, which may be observed through the use of a fluoroscope, are one exemplary type of marker. Because catheters intended for placement in the subarachnoid space are relatively thin, the associated thin marker rings can be difficult to visualize with a fluoroscope. Marker tips, which include more radiopaque material than thin marker rings, are frequently preferred because they are more susceptible to visualization with a fluoroscope. During positioning, the stylet engages the marker tip and pushes the distal portion of the catheter by applying force to the marker tip.

The present inventor has determined that it is difficult to fixedly secure a marker tip to the distal end of a catheter that is soft and thin enough for the subarachnoid space. As such, the stylet may separate the marker tip from the distal end of the subarachnoid catheter as the stylet is pushing the distal portion of the catheter to the target location within the subarachnoid space.

### SUMMARY

A catheter in accordance with one implementation of a present invention includes an abutment located within the catheter body lumen proximal to a marker tip. The abutment prevents the stylet from engaging the marker tip and/or transfers the force from the stylet to the catheter. As such, the abutment prevents the stylet from separating the marker tip from the distal end of the catheter as the stylet is pushing the distal portion of the catheter to the target location.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a representation of an implantable infusion device with a catheter that is located within the subarachnoid space.

FIG. 2 is a section view of a catheter that is located with the subarachnoid space.

FIG. 3 is a perspective view of a catheter in accordance with one embodiment of a present invention.

FIG. 4 is a section view taken along line 4-4 in FIG. 3.

FIG. 5 is an exploded section view of the catheter illustrated in FIG. 3.

FIG. 6 is a section view showing the catheter illustrated in FIGS. 3-5 in combination with a stylet.

FIG. 7 is a section of a catheter in accordance with one embodiment of a present invention.

FIG. 8 is a section view showing the catheter illustrated in FIG. 7 prior to completion.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to the exemplary implantable infusion devices described herein and, instead, are applicable to other implantable or otherwise ambulatory infusion devices that currently exist or are yet to be developed.

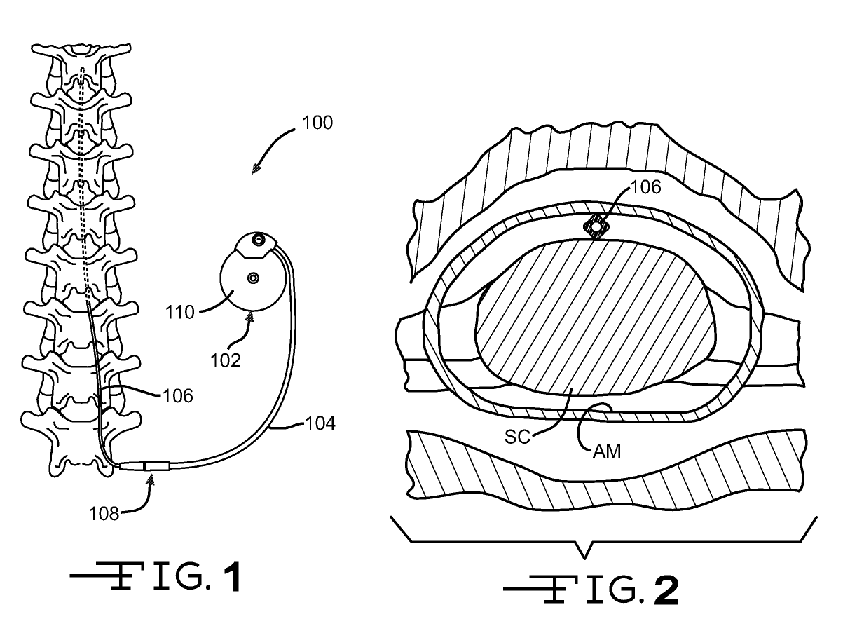

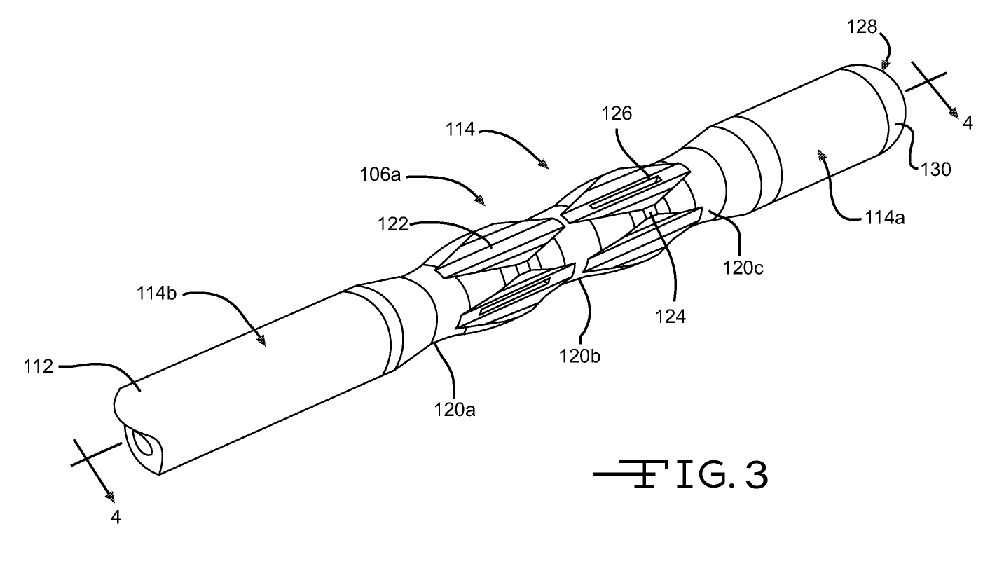

One example of an implantable infusion device in accordance with a present invention is generally represented by reference numeral 100 in FIG. 1. The implantable infusion device 100 includes an implantable pump 102, a proximal catheter 104 that is connected to the pump, a subarachnoid catheter 106, and a connector assembly 108. The implantable pump 102 includes a housing 110. An infusible substance reservoir, a fluid transfer device, control electronics and various other devices are carried within the housing 110. Although the present inventions are not limited to any particular type of implantable pump, exemplary pumps are described in U.S. Patent Pub. Nos. 2005/0273083 and 2006/0270983, which are incorporated herein by reference. The connector assembly 108 may be used to connect the proximal catheter 104 to the subarachnoid catheter 106 after the subarachnoid catheter has been positioned within the patient's body. For example, in those instances where a stylet is used to push the distal portion of the subarachnoid catheter 106 to the target location, the subarachnoid catheter will be connected to the proximal catheter 104 after the stylet has been removed. The infusible substance may then be delivered to, for example, the portion of the subarachnoid space along the spine between the spinal cord SC and the arachnoid mater AM, as is illustrated in FIG. 2.

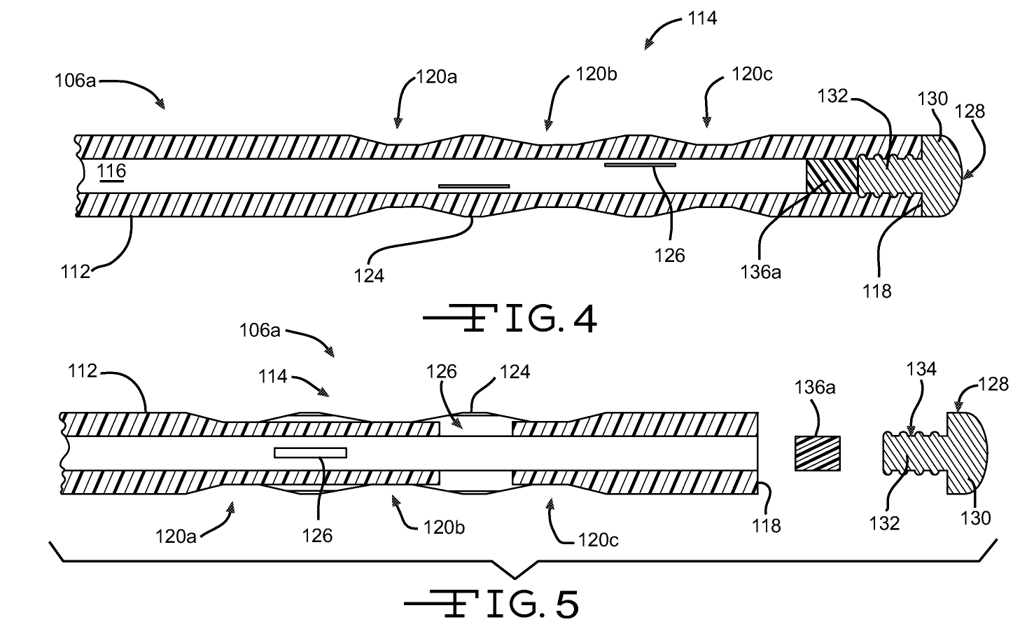

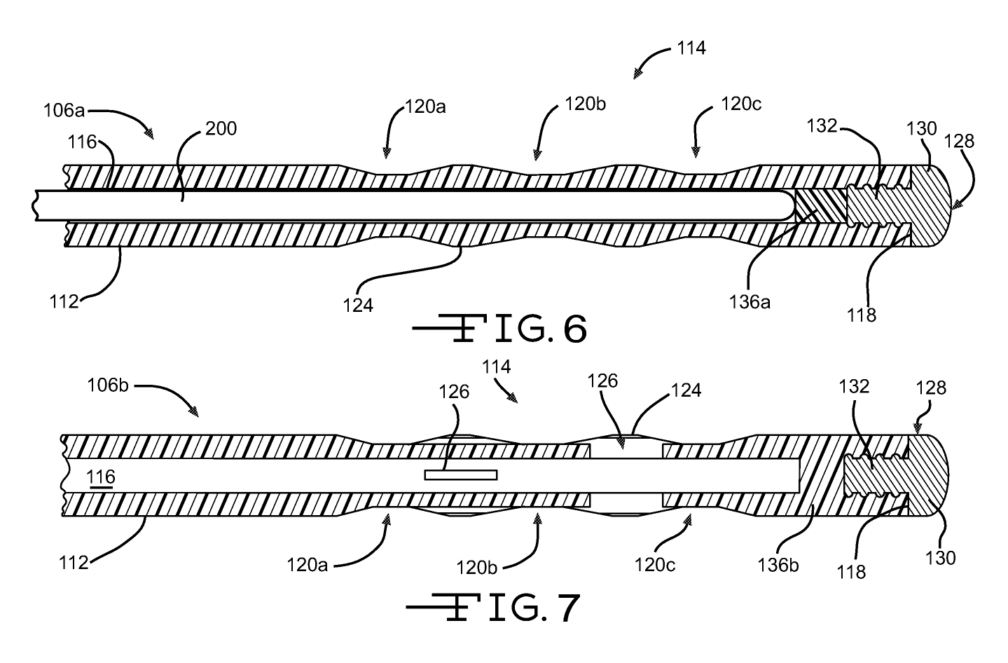

As is discussed in greater detail below, the subarachnoid catheter 106 may be configured in such a manner that a stylet will not engage the marker tip carried by the catheter. The force associated with the stylet, or at least a substantial portion of the stylet force, will be absorbed by the catheter body instead of the marker tip. A wide variety of catheters may be configured in this manner, and exemplary catheters are described below with reference to FIGS. 3-8.

Turning first to FIGS. 3-5, one example of a subarachnoid catheter is generally represented by reference numeral 106a. The subarachnoid catheter 106a includes a catheter body 112 with a distal portion 114 and a central lumen 116 that extends from the proximal end of the catheter (i.e. the end adjacent to the connector assembly 108 in FIG. 1) to the distal end 118 of the catheter. The catheter distal portion 114 includes a plurality of exterior flow regions 120a-c which have a perimeter, i.e. a circumference in the illustrated embodiment, that is smaller than that of adjacent regions of the distal portion. A plurality of slots 122 are located between the flow regions 120a-c, as are a plurality of protrusions 124. The distal portion 114 also includes a plurality of apertures 126 that extend from the exterior of the distal portion to the central lumen 116. So configured, cerebrospinal fluid (CSF) will be free to flow along the exterior of the catheter distal portion 114 from one flow region 120a-c to another, as well as in and out of the apertures 126, when distal portion regions 114a and 114b are in contact with tissue. Such flow of CSF, which is the result of the movement of the spine and beating of the heart, dilutes medication within the lumen 116 and apertures 126 that may be in contact with the arachnoid mater for prolong periods. Thus, the configuration of the distal portion 114 reduces the likelihood that granulomas, which may be due to prolonged exposure of the arachnoid mater to high concentration drugs, will form.

In the illustrated embodiment, the apertures 126 are rectangular in shape and are located in some of the slots 122. More specifically, there are four slots 122 and two diametrically opposed apertures 126 located between the flow regions 120a and 120b as well as four slots and two diametrically opposed apertures between the flow regions 120b and 120c. The apertures 126 between the flow regions 120b and 120c are offset from apertures between flow regions 120a and 120b by ninety degrees. It should be noted here, however, that the shape, number and location of the apertures 126 may be varied as desired, as may the shape, number and location of the flow regions 120a-c and slots 122. By way of example, but not limitation, the apertures 126 may be circular in shape and/or may be located in the flow regions 120a-c instead of the slots 122. In other implementations, the flow regions and slots may be eliminated. Here, the catheter body will simply be an tubular body with apertures of any suitable number, size and shape in the distal region.

A marker tip 128 is carried on the distal end 118 of the catheter body 112. The exemplary marker tip 128 is radiopaque and includes a main portion 130 and a connector 132. The connector 132, which is located within the central lumen 116, has a plurality of indentations 134 such as, for example, the illustrated plurality of longitudinally spaced concentric grooves. As is discussed below, the catheter distal portion 114 may be heated to its melting point after the marker tip connector 132 has been inserted into the central lumen 116 so that catheter material will flow into the indentations 134. In exemplary heating processes, hot air may be used to heat the catheter distal portion 114 and/or heat shrink tubing (e.g. polyimide or Teflon heat shrink tubing) may be positioned around the exterior of the catheter distal portion to control the catheter shape during the melting process. A mandrel (not shown) will also be inserted into the central lumen 116 proximal to the marker tip 128 prior to heating. The catheter material within the indentations 134, once cooled, secures the marker tip 128 to the catheter body 112. In other implementations, the connector may be smooth and secured to the catheter distal portion 114 with an adhesive. In still other implementations, marker tips may be configured such that they can be mounted on the catheter body distal end 118, and cover the distal end of the central lumen 116, without a connector that extends into the central lumen.

The exemplary subarachnoid catheter 106a illustrated in FIGS. 3-5 is also provided with an abutment 136a that is located within the central lumen 116 proximal to the marker tip 128. The exemplary abutment 136a, which is cylindrical in shape and has an outer diameter (OD) that is equal to, or is about 0.001 to 0.003 inches greater than, the inner diameter (ID) of the catheter body 112, may be formed from any suitable material and secured to the catheter distal portion 114 with an adhesive. The abutment 136a may also be located directly proximal of the marker tip 128 (as shown) or may be proximally spaced therefrom by a short distance (e.g. about 0.040 inch). Turning to FIG. 6, the abutment 136a performs the function of preventing a stylet 200 from engaging the marker tip 128. The abutment 136a also performs the function of transferring the force from the stylet 200 to the catheter body distal portion 114 instead of the marker tip 128. As such, the stylet 200 will not separate the marker tip 128 from the distal end 118 of the catheter body 112 as the stylet is pushing the distal portion 114 of the catheter to a target location within, for example, the subarachnoid space around the spinal cord.

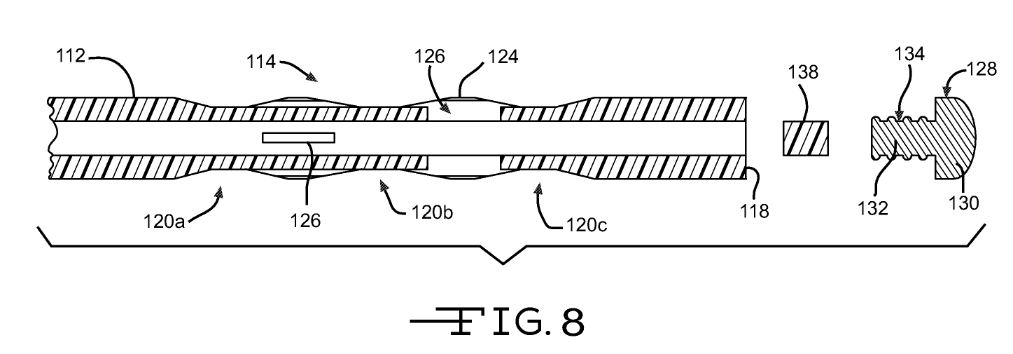

Another exemplary subarachnoid catheter is generally represented by reference numeral 106b in FIG. 7. Subarachnoid catheter 106b is substantially similar to subarachnoid catheter 106a and similar elements are represented by similar reference numerals. Here, however, the abutment 136b is an integral part of the catheter body distal portion 114 as opposed to a separate structure that is secured to the distal portion with, for example, an adhesive. The integral abutment 136b, which performs the same functions as abutment 136a, may be formed in a variety of ways. For example, a donor structure 138, such as a cylindrical donor structure that is formed from the same material as the catheter body 112 and has an OD that corresponds to the ID of the catheter body, may be inserted into the lumen 116, as is illustrated in FIG. 8. The catheter body 112 and donor structure 138 may then be heated to the melting temperature of the material. A mandrel (not shown) will also be inserted into the central lumen 116 proximal to the donor structure 138 prior to heating. The catheter body distal portion 114 and donor structure 138 will merge and, once cooled, will provide the catheter body distal portion with the integral abutment 136b illustrated in FIG. 7. In those instances where the marker tip 128 is secured to the catheter body 112 by heating the catheter distal portion 114 to its melting point after the marker tip connector 132 has been inserted into the central lumen 116, the marker tip may be secured to the catheter body while the integral abutment is being formed.

Abutments may be formed in other ways. By way of example, but not limitation, an abutment may be formed by injecting a hardenable material into the central lumen 116. An adhesive, such as room temperature vulcanizing (RTV) silicone rubber, is one example of a suitable hardenable material.

With respect to materials, suitable materials for the catheter body 112 include, but are not limited to polymers such as polyurethane (e.g. Carbothane® 95A), silicone, polyethylene, and polypropylene. Suitable materials for the marker tip 128 include, but are not limited to, radiopaque materials such as platinum, gold, tungsten and iridium. With respect to dimensions, the exemplary catheter body 112, which is configured for use in the subarachnoid space, is circular in cross-section and has an OD of about 0.055 inches and an ID of about 0.021 inches. The OD at the exterior flow regions 120a-c is about 0.042 inches, and adjacent exterior flow regions are about 0.1 inch apart. The present catheters are not, however, limited to a circular cross-sectional shape. The length of the catheter body 112 may also vary from about 10 inches to about 40 inches, depending on the intended application.

Although the inventions disclosed herein have been described in terms of the preferred embodiments above, numerous modifications and/or additions to the above-described preferred embodiments would be readily apparent to one skilled in the art. By way of example, but not limitation, the present inventions are applicable to catheters that supply stimulation energy, as opposed to or in addition to, infusible substances. Such catheters are sometimes referred to a spinal cord stimulation leads. It is intended that the scope of the present inventions extend to all such modifications and/or additions and that the scope of the present inventions is limited solely by the claims set forth below.

1. An apparatus, comprising:
a tubular catheter body defining a distal portion, a distal end and a lumen that extends to the distal end;
a substantially radiopaque marker tip carried by the distal end of the catheter body such that a portion of the marker tip is aligned with the lumen; and
an abutment located within the lumen proximal to the substantially radiopaque marker tip.

2. An apparatus as claimed in claim 1, wherein the tubular catheter body defines an exterior surface and includes at least one aperture that extends from the exterior surface to the lumen.

3. An apparatus as claimed in claim 1, wherein the tubular catheter body defines a size and a shape that facilitates insertion into an intrathecal space.

4. An apparatus as claimed in claim 1, wherein the substantially radiopaque marker tip includes a connector that is located within the lumen.

5. An apparatus as claimed in claim 4, wherein the connector includes a plurality of indentations and portions of the tubular catheter body are located within the indentations.

6. An apparatus as claimed in claim 5, wherein the plurality of indentations comprises a plurality of concentric grooves.

7. An apparatus as claimed in claim 1, wherein the abutment is integral with the catheter body.

8. An apparatus as claimed in claim 1, wherein the abutment comprises a cylindrical structure secured to the catheter body.

9. An apparatus as claimed in claim 1, wherein the distal portion of the catheter body includes at least one exterior groove and/or at least one exterior flow region.

10. An apparatus for use with a stylet, comprising:
a tubular catheter body defining a distal portion, a distal end and a lumen that extends to the distal end;
a substantially radiopaque marker tip carried by the distal end of the catheter body such that a portion of the marker tip is aligned with the lumen; and
means, associated with the distal portion of the tubular catheter body, for preventing the stylet from engaging the substantially opaque marker tip.

11. An apparatus as claimed in claim 10, wherein the tubular catheter body defines an exterior surface and includes at least one aperture that extends from the exterior surface to the lumen.

12. An apparatus as claimed in claim 10, wherein the tubular catheter body defines a size and a shape that facilitates insertion into an intrathecal space.

13. An apparatus as claimed in claim 10, wherein the substantially radiopaque marker tip includes a connector that is located within the lumen.

14. An apparatus as claimed in claim 13, wherein the connector includes a plurality of indentations and portions of the tubular catheter body are located within the indentations.

15. An apparatus as claimed in claim 14, wherein the plurality of indentations comprises a plurality of concentric grooves.

16. An apparatus as claimed in claim 10, wherein the distal portion of the catheter body includes at least one exterior groove and/or at least one exterior flow region.

17. An apparatus for use with a stylet, comprising:
a tubular catheter body defining a distal portion, a distal end and a lumen that extends to the distal end;
a substantially radiopaque marker tip carried by the distal end of the catheter body such that a portion of the marker tip is aligned with the lumen; and
means, associated with the distal portion of the tubular catheter body, for transferring force associated with the stylet to the catheter body distal portion.

18. An apparatus as claimed in claim 17, wherein the tubular catheter body defines an exterior surface and includes at least one aperture that extends from the exterior surface to the lumen.

19. An apparatus as claimed in claim 17, wherein the tubular catheter body defines a size and a shape that facilitates insertion into an intrathecal space.

20. An apparatus as claimed in claim 17, wherein the substantially radiopaque marker tip includes a connector that is located within the lumen.

21. An apparatus as claimed in claim 20, wherein the connector includes a plurality of indentations and portions of the tubular catheter body are located within the indentations.

22. An apparatus as claimed in claim 21, wherein the plurality of indentations comprises a plurality of concentric grooves.

23. An apparatus as claimed in claim 17, wherein the distal portion of the catheter body includes at least one exterior groove and/or at least one exterior flow region.

24-25. (canceled)

26. An apparatus as claimed in claim 1, wherein the abutment comprises an injectable and hardenable material.

27. An apparatus as claimed in claim 26, wherein the injectable and hardenable material comprises an adhesive.

28. An apparatus as claimed in claim 1, wherein the distal end defines a distal-most surface of the tubular catheter body and the lumen extends through the distal-most surface.

29. An apparatus as claimed in claim 28, wherein a portion of the substantially radiopaque marker tip abuts the distal-most surface.

30. An apparatus as claimed in claim 1, wherein
the lumen defines a longitudinal axis; and
the substantially radiopaque marker tip is located on the longitudinal axis.

31. An apparatus as claimed in claim 1, wherein
the abutment defines a distal end; and
a portion of the lumen is distal of the distal end of the abutment.

32. An apparatus as claimed in claim 1, wherein the abutment and the catheter are formed from different materials.

33. An apparatus as claimed in claim 10, wherein the distal end defines a distal-most surface of the tubular catheter body and the lumen extends through the distal-most surface.

34. An apparatus as claimed in claim 33, wherein a portion of the substantially radiopaque marker tip abuts the distal-most surface.

35. An apparatus as claimed in claim 10, wherein
the lumen defines a longitudinal axis; and
the substantially radiopaque marker tip is located on the longitudinal axis.

36. An apparatus as claimed in claim 10, wherein
the abutment defines a distal end; and
a portion of the lumen is distal of the distal end of the abutment.

37. An apparatus as claimed in claim 10, wherein the abutment and the catheter are formed from different materials.
